(19) United States
(12) Patent Application Publication
(10) Pub. No.: US 2024/0315655 A1
(43) Pub. Date: Sep. 26, 2024

(54) A METHOD FOR CEREBRAL VESSEL CALCIFICATION DETECTION AND QUANTIFICATION, USING MACHINE LEARNING

(71) Applicant: Inteneural Networks Inc., Chicago, IL (US)

(72) Inventors: Kris B, SIEMIONOW, Chicago, IL (US); Paul LEWICKI, Tulsa, OK (US)

(21) Appl. No.: 18/574,119

(22) PCT Filed: Jun. 27, 2022
(86) PCT No.: PCT/US2022/035066

§ 371 Date: Dec. 26, 2023

Related U.S. Application Data
(60) Provisional application No. 63/214,949, filed on Jun. 25, 2021.

(51) Int. Cl. A61B 6/50; A61B 6/00; A61B 6/03; G06T 7/00; G06T 7/11

(52) U.S. Cl. CPC A61B 6/504; A61B 6/501; A61B 6/5217; G06T 7/0014; G06T 7/11; A61B 6/032; G06T 2207/10081; G06T 2207/20081; G06T 2207/20084; G06T 2207/30016; G06T 2207/30101

(57) ABSTRACT

A method for cerebral vessel calcification detection and classification includes the steps of: receiving a set of input Computed Tomography (CT) images representing consecutive slices of a 3D volume of cerebral vessels; performing a region of interest regression to determine a ROI within the input CT images that is a cuboid that contains a circle of Willis; performing calcification detection based on the ROI within the input CT images, by means of: a segmentation procedure and/or an anomaly detection procedure; and performing quantification of the predicted locations of vessel calcifications to indicate at least one of: a volume or intensity of individual calcifications.

### TECHNICAL FIELD

The present invention relates to detection and quantification of cerebral vessel calcifications by use of machine learning methods.

### BACKGROUND

Ischemic stroke is a major challenge affecting many developed societies. It is caused by multiple factors including vascular wall pathology in carotid and intracranial vessels. Gradual growth of plaques (calcifications) within the cerebral vessels impairs the flow and enables adhesion of platelets. Both processes impair the cerebral blood flow and can cause brain ischemia. The process of vascular injury affects both extracranial and intracranial vessels, yet it is different in smaller vessels. Development of pathology in carotid vessels is often promoted by geometry of the carotid vessels, however in distal vessels where the flow is slower the main mechanism behind development of pathology remains elusive. In the process of plaque formation initially the lesions are invisible on Computed Tomography (CT) images however over time with the accumulation of calcified components it becomes detectable on non-contrast computed tomography.

The pathophysiology of the plaques is dynamic and multiple mechanisms might contribute to final tissue injury; therefore, it is important to detect and quantify any potential cause that is treatable using currently available therapies like anticoagulants targeting hypercoagulable states.

The mechanism of stroke is often elusive because the causing factor might already be absent when the permanent tissue injury of the brain is evaluated using neuroimaging methods. This is often the case with transient cerebral hypoperfusion caused by embolism, obstruction of the flow and impaired oxygen carrying capacity.

### SUMMARY OF THE INVENTION

Approximately one third of patients diagnosed with acute ischemic stroke have undetermined cause of the disease. This is negatively affecting the secondary stroke prevention. To improve secondary stroke prevention, it is proposed to detect and quantify the intravascular calcium load in the intracranial arteries starting with the intracranial portion of the internal carotid artery and its distal branches i.e., middle cerebral artery and anterior cerebral artery, as well as vertebral arteries and basilar artery and its distal branches i.e., posterior cerebral arteries. Pathophysiology of the impaired perfusion might be affected by the geometry of the atherosclerotic intracranial plaque. Various shapes can be identified: ring-like shapes, cylindrical shapes and fragments of either one.

There is a need for a method to detect and quantify intra-arterial plaques located in the intracranial vessels.

In one aspect, the invention relates to a method for cerebral vessel calcification detection and classification, the method comprising the steps of: receiving a set of input Computed Tomography (CT) images representing consecutive slices of a 3D volume of cerebral vessels; performing a region of interest regression to determine a ROI within the input CT images that is a cuboid that contains a circle of Willis; performing calcification detection based on the ROI within the input CT images, by means of: a segmentation procedure that comprises using a segmentation neural network to perform segmentation of the ROI of the input CT images and output a binary mask denoting predicted locations of vessel calcifications, wherein the segmentation neural network is trained by a training set comprising ROI of CT images of cerebral vessels with calcifications as input and corresponding binary masks denoting the calcifications as output; and/or an anomaly detection procedure that comprises using an anomaly detection neural network to perform analysis of the ROI of the input CT images and output a binary mask denoting detected areas that are predicted as different from a healthy area as predicted locations of vessel calcifications, wherein the anomaly detection neural network is trained by a training set comprising ROI of CT images of cerebral vessels of healthy brains as both input and output; and performing quantification of the predicted locations of vessel calcifications to indicate at least one of: a volume or intensity of individual calcifications.

The method may further comprise converting the input CT images to a bone window.

The ROI may contain a volume that is in each direction 25% larger than the maximum circle of Willis size in each direction.

The step of performing the region of interest regression can be performed by a ROI extraction neural network.

The ROI extraction neural network may comprise an input convolutional neural network feature extractor with 3D convolutions configured to recover essential features necessary for ROI placement and an output fully connected stage with outputs which define the predicted position of the ROI based on the essential features.

The ROI can be defined by spatial coordinates of a center of the ROI and spatial size of the ROI in each direction.

The ROI can be defined by spatial coordinates of a center of the ROI and a predefined size for each set of input images.

The method may comprise performing both the segmentation procedure (203A) and the anomaly detection procedure (203B).

In another aspect, the invention is related to a computer-implemented system, comprising at least one nontransitory processor-readable storage medium that stores at least one of processor-executable instructions or data; and at least one processor communicably coupled to at least one nontransitory processor readable storage medium, wherein at least one processor is configured to perform the steps of the method as described herein.

The presented system and method are useful for estimating cerebral plaques in intracerebral vessels using CT imaging as input data. An output image volume is generated comprising at least one vessel, with generated and quantified location, size, volume and number of calcified deposits. The output can be visualized so as to allow better assessment of lesions.

These and other features, aspects and advantages of the invention will become better understood with reference to the following drawings, descriptions and claims.

### BRIEF DESCRIPTION OF DRAWINGS

Various embodiments are herein described, by way of example only, with reference to the accompanying drawings, wherein:

FIGS. 1A and 1B show a non-contrast computed tomography of the head;

FIG. 1C shows schematically the circle of Willis and a region of interest;

FIG. 1D shows the region of interest marked on an input image;

FIG. 2 shows a method for detecting calcifications;

FIG. 3 shows a ROI extraction neural network;

FIG. 4 shows a segmentation neural network;

FIG. 5 shows an anomaly detection neural network:

FIG. 6 shows a structure of a computer system for implementing the method of FIG. 2.

### DETAILED DESCRIPTION

The following detailed description is of the best currently contemplated modes of carrying out the invention. The description is not to be taken in a limiting sense but is made merely for the purpose of illustrating the general principles of the invention.

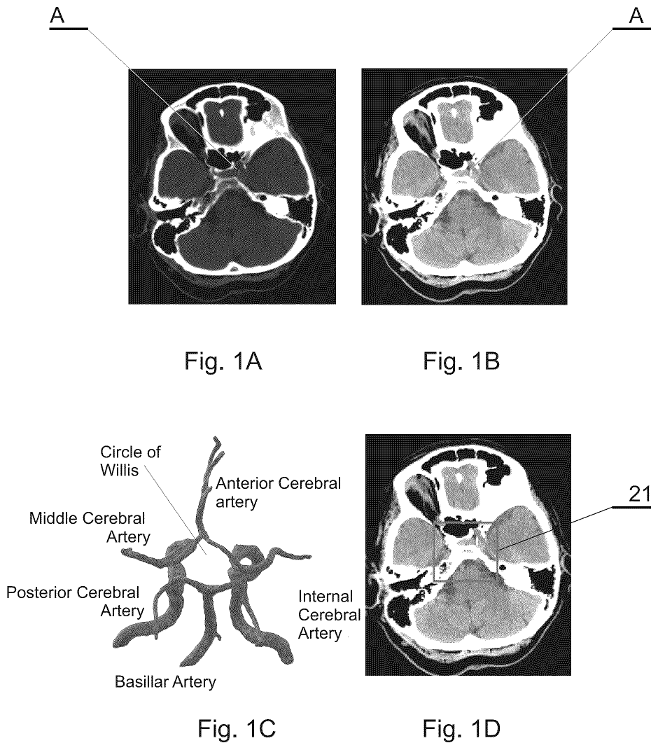

FIGS. 1A and 1B show a non-contrast computed tomography of the head made for different HU (Hounsfield Unit) values-FIG. 1A is presented in brain window and FIG. 1B close to the bone window. On these images, presence of small vessels calcification A can be identified in the terminal portion of the left and right internal carotid arteries. Identification of such calcifications can help in diagnosis of intracranial stenosis and careful escalation of the therapy aimed at preventing ischemic stroke.

In another embodiment, as an alternative to non-contrast computed tomography images of FIGS. 1A and 1B, contrast-enhanced CT images can be used as well.

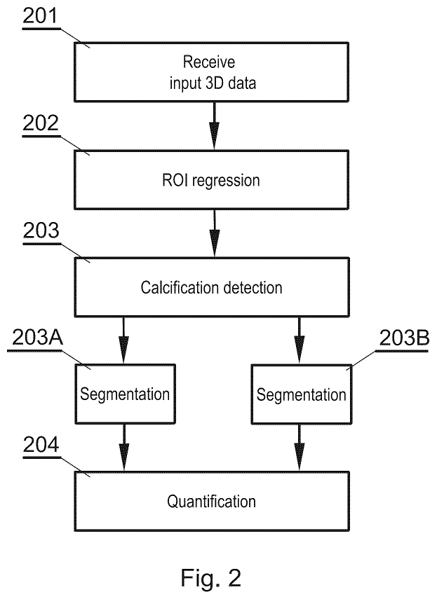

The calcifications are detected by a method shown in FIG. 2. First, a set of input images is received in step 201 that are non-contrast CT images or contrast-enhanced CT images and that represent consecutive slices of a 3D volume of cerebral vessels. Preferably, the input images are converted to the bone window so that the calcifications can be easier distinguished.

Next, the calcification detection is a two-stage process. The first step 202 is the region of interest regression, and the second step 203 is the calcification detection.

The first step 202, namely the regression of the coordinates of the region of interest is performed using a ROI extraction neural network 300. The neural network allows to efficiently detect ROI within images from different sources, namely different machine types, patients (who may be slightly differently positioned) and protocols. The aim of the step 202 is to place a region of interest that is a cuboid that constrains the area of operation of the subsequent processing to the volume that contains the circle of Willis, and preferably about 25% more than the maximum circle of Willis size in each direction (such ROI 21 is shown schematically in FIG. 1C).

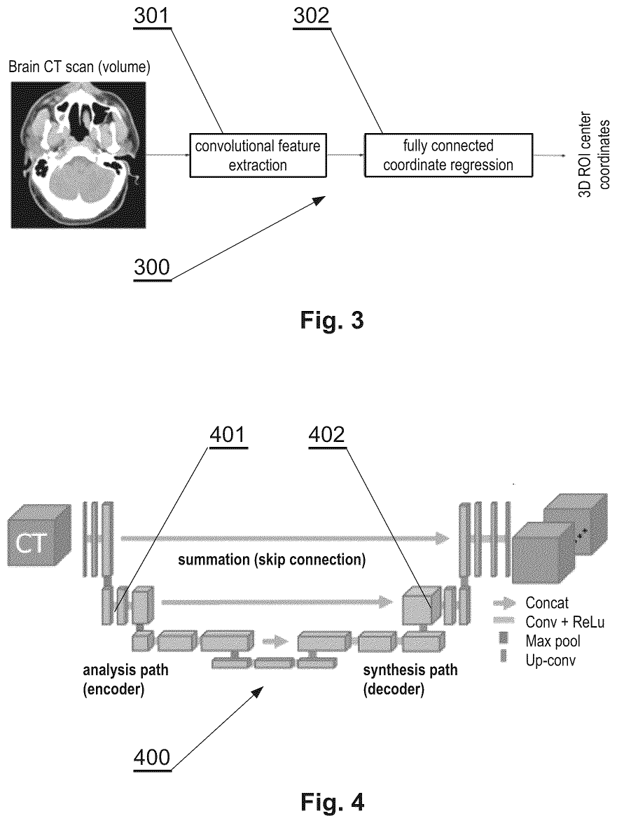

The ROI extraction neural network 300 shown in FIG. 3 estimates the 3D coordinates of the center of the fixed size cuboid region of interest from the complete volume of the brain received as input data. The ROI extraction neural network 300 is composed of an input convolutional neural network feature extractor 301 using 3D convolutions to recover the essential features necessary for region of interest placement and an output fully connected stage 302 with outputs which define the predicted position of the region of interest based on the essential features (for example, by specifying the x, y, z coordinates of its center and optionally the size in the x, y, z directions (or, the size can be assumed to have predetermined values)). The illustration of ROI placement on a slice of an actual input image is shown in FIG. 1D.

The ROI extraction neural network 300 is trained using the input 3D volume of the CT-scanned brain on its input and the sets of ground truth 3D ROI coordinates at its output. Upon successful training of the network, it can be used to predict the coordinates for ROI placement on previously unseen scans.

Subsequently, the extracted ROI 21 is the input volume for the subsequent step 203 of the procedure using a segmentation and/or anomaly detection neural network.

The calcification detection in step 203 can be performed using two approaches segmentation in step 203A and/or anomaly detection in step 203B based on the ROI 21.

The segmentation of step 203A can be performed by means of a segmentation neural network 400 shown in FIG. 4 that is a 3D Unet-like structure. The segmentation neural network 400 architecture contains two paths 401, 402. The first path 401 is the contraction path (also called as the encoder) which is used to capture the context in the volume. The encoder 401 is a convolutional neural network that performs the task of characteristic feature extraction based on local context using a hierarchy of increasingly complex features. The spatial resolution of features is progressively contracted, and their number increases in the subsequent layers of the network. The encoder can have an architecture of a neural network such as VGG, ResNet or EfficientNet. The encoder can be pre-trained, that is trained to perform binary segmentation for a similar task in the same domain.

The second path 402 is the symmetric expanding path (also called as the decoder) which is used to enable precise localization using transposed convolutions. The corresponding levels of the encoder and the decoder are connected, which enables feature sharing. The encoder-decoder connections can be denser, such as presented by the Unet++ neural network architecture. The decoder can be supplemented with additional trainable attention modules.

The segmentation neural network 400 is trained using 3D volumes restricted to the previously extracted ROI of the CT-scanned brain having cerebral vessels with calcifications and corresponding binary masks denoting the vessel calcifications within the input 3D volumes. Upon successful training of the segmentation neural network 400, it can be used to predict the location of calcifications in the extracted ROI of a previously unseen scan. Therefore, the segmentation neural network 400 outputs a binary mask denoting predicted locations of vessel calcifications.

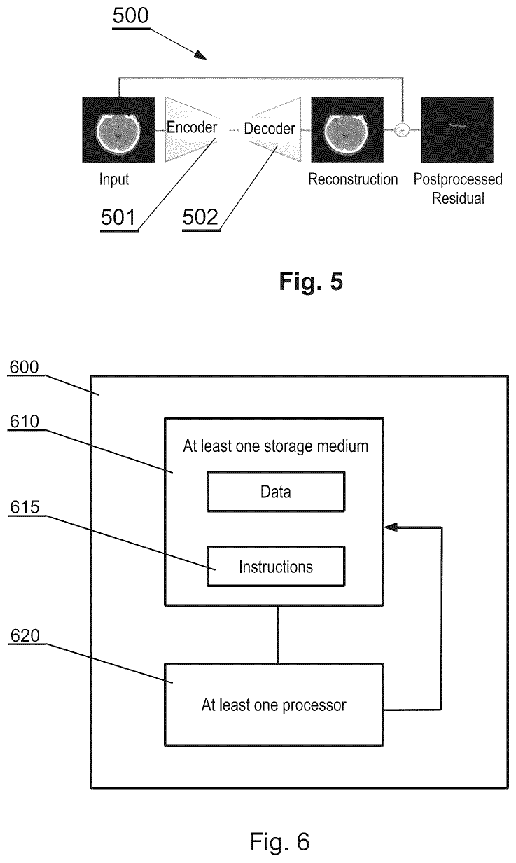

The anomaly detection can be performed by an anomaly detection neural network 500 shown in FIG. 5.

The anomaly detection neural network 500 is an encoder-decoder neural network trained using a dataset consisting of normal (healthy) cases to reconstruct the input image at its output will in general overfit to the distribution used for training. The encoder is a convolutional neural network which extracts internal representation of the input image to a more compact form that still enables reconstruction with a trained decoder. The decoder is responsible for deconvolution and un-pooling/up-sampling to perform final pixel-wise predictions forming the resulting image. The network is trained with a large set of healthy brain images to return the same images on its output. The network will therefore be biased towards generating brain images that appear healthier. With a “healthy” brain on the input, the output image will differ only slightly with respect to the input image. Comparing the differences in volume will yield no regions of interest. With an “unhealthy brain” on its input, the neural network will make a prediction so that the output image or volume will look more like a healthy brain, since it was trained to be biased and try to do that with every sample. The regions that differ between the input and the predicted output image will be anomalous regions, which appear “unhealthy”. As a result, presenting an “unhealthy” example to the neural network input will generate a residual volume, in which the anomalous regions are highlighted. Therefore, using a dataset of healthy brains in which calcifications are not present the network can be trained to highlight the regions which differ from the healthy ones—the regions in which the calcifications are present. Therefore, the anomaly detection neural network 500 outputs a binary mask denoting detected areas that are predicted as different from a healthy area. The advantage of this approach is that it does not require binary mask annotations for training, as compared to the segmentation neural network approach (but it can be less accurate).

Both methods can be used simultaneously, as they are fully independent. In one embodiment, results of both methods can be shown independently. In another embodiment, a logical conjunction or alternative of results of both methods can be provided as an output.

Next, in step 204, the detected calcifications are quantified in order to accurately consider potential treatment and available therapies. For example, the following set of features can be quantified. First, the volume of individual calcifications is computed, for example by multiplying the number of voxels classified as containing calcification by voxel spacing which is available in the imaging data format. Similarly, this spacing information combined with shape of object indicated by calcification mask can be used to compute its dimensions, namely length of its longest axis and perpendicular to the longest axis. Intensity of the calcification can also be measured using the previously obtained binary mask of detected calcification to select corresponding voxels from the original raw input and computing their median and interquartile range. The finding can be classified into mild, moderate and severe identifies intracranial vessel wall calcifications associated with vessel stenosis, hypoperfusion and increased risk of atherosclerosis.

The functionality described herein can be implemented in a computer-implemented system 600, such as shown in FIG. 6. The system may include at least one non-transitory processor-readable storage medium that stores at least one of processor-executable instructions or data and at least one processor communicably coupled to at least one non-transitory processor-readable storage medium. At least one processor is configured to perform the steps of the methods presented herein. The computer-implemented system 600, for example a machine-learning system, may include at least one non-transitory processor-readable storage medium 610 that stores at least one of processor-executable instructions 615 or data; and at least one processor 620 communicably coupled to the at least one non-transitory processor-readable storage medium 610. At least one processor 620 may be configured to (by executing the instructions 615) to perform the steps of the method of FIG. 2.

While the invention has been described with respect to a limited number of embodiments, it will be appreciated that many variations, modifications and other applications of the invention may be made. Therefore, the claimed invention as recited in the claims that follow is not limited to the embodiments described herein.

1. A method for cerebral vessel calcification detection and classification, the method comprising the steps of:
receiving a set of input Computed Tomography (CT) images representing consecutive slices of a 3D volume of cerebral vessels;
performing a region of interest regression to determine a ROI within the input CT images that is a cuboid that contains a circle of Willis;
performing calcification detection based on the ROI within the input CT images, by means of:
a segmentation procedure that comprises using a segmentation neural network to perform segmentation of the ROI of the input CT images and output a binary mask denoting predicted locations of vessel calcifications, wherein the segmentation neural network is trained by a training set comprising ROI of CT images of cerebral vessels with calcifications as input and corresponding binary masks denoting the calcifications as output; and/or
an anomaly detection procedure that comprises using an anomaly detection neural network to perform analysis of the ROI of the input CT images and output a binary mask denoting detected areas that are predicted as different from a healthy area as predicted locations of vessel calcifications, wherein the anomaly detection neural network is trained by a training set comprising ROI of CT images of cerebral vessels of healthy brains as both input and output;
and performing quantification of the predicted locations of vessel calcifications to indicate at least one of: a volume or intensity of individual calcifications.

2. The method according to claim 1, further comprising converting the input CT images to a bone window.

3. The method according to claim 1, wherein the ROI contains a volume that is in each direction 25% larger than the maximum circle of Willis size in each direction.

4. The method according to claim 1, wherein the step of performing the region of interest regression is performed by a ROI extraction neural network.

5. The method according to claim 4, wherein the ROI extraction neural network comprises an input convolutional neural network feature extractor with 3D convolutions configured to recover essential features necessary for ROI placement and an output fully connected stage with outputs which define the predicted position of the ROI based on the essential features.

6. The method according to claim 1, wherein the ROI is defined by spatial coordinates of a center of the ROI and spatial size of the ROI in each direction.

7. The method according to claim 1, wherein the ROI is defined by spatial coordinates of a center of the ROI and a predefined size for each set of input images.

8. The method according to claim 1, comprising performing both the segmentation procedure (203A) and the anomaly detection procedure (203B).

9. A computer-implemented system, comprising at least one nontransitory processor-readable storage medium that stores at least one of processor-executable instructions or data; and at least one processor communicably coupled to at least one nontransitory processor readable storage medium, wherein at least one processor is configured to perform the steps of the method according to any of previous claims.
